(19) United States
(12) Patent Application Publication
(10) Pub. No.: US 2011/0108767 A1
(43) Pub. Date: May 12, 2011

(54) COMPOSITION, AND LIGHT-EMISSION ELEMENT PRODUCED BY USING THE COMPOSITION

(75) Inventor: Nobuhiko Akino, Tsukuba (JP)

(73) Assignees: Sumitomo Chemical Company, Limited; Sumation Co., Ltd.

(21) Appl. No.: 13/000,521

(22) PCT Filed: Jun. 23, 2009
(86) PCT No.: PCT/JP2009/061362

§ 371 Date: Dec. 21, 2010

(30) Foreign Application Priority Data
Jun. 23, 2008 (JP) 2008-163039

(51) Int. Cl. C09K 11/06; C08F 226/06

(52) U.S. Cl. 252/301.16; 526/258

(57) ABSTRACT

Disclosed is a composition comprising a compound having a pyridazine ring structure and a phosphorescent compound.

### TECHNICAL FIELD

The present invention relates to a composition and a light-emitting device prepared by using the composition.

### BACKGROUND ART

As a light-emitting material for use in a light-emitting layer of a light-emitting device, a compound emitting light from a triplet excitation state (hereinafter, sometimes referred to as a “phosphorescent compound”) is known. The device using this compound in a light-emitting layer is known to have a high luminous efficiency. When a phosphorescent compound is used in a light-emitting layer, usually, a composition prepared by adding the compound to a matrix is used as a light-emitting material. As the matrix, a compound such as polyvinylcarbazole is used since a thin film can be formed by coating (PATENT DOCUMENT 1).

However, it is difficult to inject electrons to such compound because the level of the lowest unoccupied molecular orbital (hereinafter, referred to as the “LUMO”) thereof is high. On the other hand, a conjugated macromolecular compound such as polyfluorene has a low LUMO. Thus, if it is used as a matrix, a low driving voltage can be realized relatively easily. However, such a conjugated macromolecular compound, since the lowest triplet excitation energy thereof is low, is not suitable as a matrix used for emitting light having a shorter wavelength than that of green light, in particular (PATENT DOCUMENT 2). For example, in a light-emitting material composed of polyfluorene as a conjugated macromolecular compound and a triplet emission compound (NON-PATENT DOCUMENT 1), light emission from triplet emission compound is weak. Thus, the luminous efficiency thereof is low.

### CITATION LIST

### Patent Document

PATENT DOCUMENT 1: JP 2002-50483 A
PATENT DOCUMENT 2: JP 2002-241455 A

### Non-Patent Document

NON-PATENT DOCUMENT 1: APPLIED PHYSICS LETTERS, 80, 13, 2308 (2002)

### SUMMARY OF THE INVENTION

### Problems to be Solved by the Invention

In the circumstances, an object of the invention is to provide a light-emitting material providing an excellent luminous efficiency when used in a light-emitting device, etc.

### Means for Solving the Problems

The present inventors have repeatedly conducted intensive studies. As a result, they have found that the aforementioned problem can be overcome by a composition comprising a compound having a pyridazine ring structure and a phosphorescent compound, and attained the present invention.

More specifically, the present invention firstly provides a composition comprising a compound having a pyridazine ring structure and a phosphorescent compound.

The present invention secondly provides a macromolecular compound having a residue of the phosphorescent compound and the pyridazine ring structure.

The present invention thirdly provides a light-emitting thin film, an organic semiconductor thin film and a light-emitting device prepared by using the composition or the macromolecular compound.

The present invention fourthly provides a planar light source, a segment display and a dot matrix display having the light-emitting device, a light having the light-emitting device and a liquid crystal display having the light-emitting device as a backlight.

### Advantages of the Invention

The composition and macromolecular compound of the present invention (hereinafter, referred to as “the composition etc. of the present invention”) has a high luminous efficiency. Therefore, when the composition etc. of the present invention is used in preparation of a light-emitting device, etc., a light-emitting device excellent in luminous efficiency can be obtained. Furthermore, the composition etc. of the present invention usually has a relatively excellent luminosity. This is because the lowest triplet excitation energy of a compound (a compound having a pyridazine ring) contained in the composition of the present invention and a macromolecular compound of the present invention is large. Furthermore, a composition having a relatively low LUMO and to which electrons are easily injected can be obtained.

### MODE FOR CARRYING OUT THE INVENTION

Next, the present invention will be more specifically described below. Note that in the specification, in the case where an alkyl group and an alkoxy group of a structural formula has no prefix (t-, etc.), they means n-.

### <Composition>

The composition of the present invention contains a compound having a pyridazine ring structure and a phosphorescent compound. In the present invention, the pyridazine ring structure refers to pyridazine, a group provided by removing all or some (1 or 2 in particular) of the hydrogen atoms of pyridazine. Furthermore, the “macromolecular compound” refers to a compound having at least two identical structures (repeating units) in the compound.

The compound having a pyridazine ring structure preferably has at least one pyridazine ring structure selected from the group consisting of the pyridazine ring structures represented by the following general formulas (1-1), (1-2), (2-1), (2-2), (2-3) and (2-4):

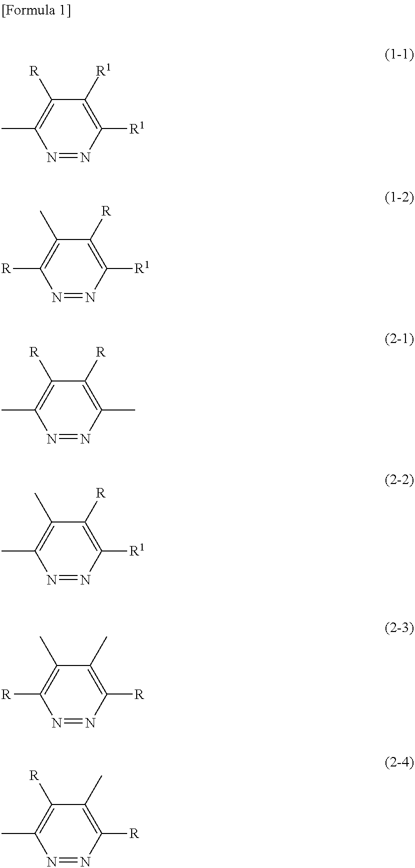

wherein, R and R¹ each independently represent a hydrogen atom or a monovalent substituent; and when there are more than one R or R¹, they may be the same or different, and more preferably has at least two pyridazine ring structures. When the compound having a pyridazine ring structure is a macromolecular compound, the macromolecular compound more preferably has the pyridazine ring structure in the main chain and/or a side chain of the macromolecular compound. Examples thereof include a macromolecular compound having a repeating unit whose structure is represented by the above general formula (2-1), (2-2), (2-3) or (2-4) in the main chain and/or a side chain and a macromolecular compound having a repeating unit whose structure is represented by the above general formula (1-1) or (1-2) in a side chain. A compound containing any structure selected from an aromatic ring, a heterocyclic ring having at least 5-members containing a hetero atom, an aromatic amine and a structure represented by the following general formula (4) in addition to the structures represented by the above general formulas (1-1), (1-2), (2-1), (2-2), (2-3) and (2-4) is particularly preferable.

In the formulas (1-1), (1-2), (2-1), (2-2), (2-3) and (2-4), R and R¹ each independently represent a hydrogen atom or a monovalent substituent, preferably, at least one of more than one R or R¹ is a monovalent substituent, and more preferably, all of more than one R or R¹ are monovalent substituents. More than one R or R¹ may be independently the same or different.

Examples of the monovalent substituent include a halogen atom, an alkyl group, an alkoxy group, an alkylthio group, an aryl group that may have a substituent, an aryloxy group, an arylthio group, an arylalkyl group, an arylalkyloxy group, an arylalkylthio group, an acyl group, an acyloxy group, an amide group, an acid imide group, an imine residue, a substituted amino group, a substituted silyl group, a substituted silyloxy group, a substituted silylthio group, a substituted silylamino group, a monovalent heterocyclic group that may have a substituent, a heteroaryl group that may have a substituent, a heteroaryloxy group, a heteroarylthio group, an arylalkenyl group, an arylethynyl group, a substituted carboxyl group and a cyano group, and preferably, include an alkyl group, an alkoxy group, an aryl group that may have a substituent and a heteroaryl group that may have a substituent. Note that the N-valent heterocyclic group (N is 1 or 2) refers to a remaining atomic group provided by removing N hydrogen atoms from a heterocyclic compound; the same will apply hereinafter. Note that as a monovalent heterocyclic group, a monovalent aromatic heterocyclic group is preferable.

At least one of the R and R¹ is preferably an alkyl group, an alkoxy group, an aryl group that may have a substituent or a heteroaryl group that may have a substituent. At least one of the R and R¹ is further preferably an alkyl group having 3 to 10 carbon atoms or an alkoxy group having 3 to 10 carbon atoms.

At least one of the R is preferably a monovalent substituent having 3 or more atoms in total except a hydrogen atom, further preferably a monovalent substituent having 5 or more atoms in total except a hydrogen atom, and particularly preferably a monovalent substituent having 7 or more atoms in total except a hydrogen atom. When two R are present, at least one of the R is preferably the monovalent substituent, and more preferably two R are the monovalent substituents. More than one R or R¹ may be independently the same or different.

As the compound having a pyridazine ring structure, a compound represented by the following general formula (3-1) or (3-2):

[Formula 2]

pdz-(Y¹)ₙ—Ar¹ (3-1)

pdz-(Y¹)ₙ-pdz (3-2)

wherein, pdz represents a pyridazine ring structure represented by the above general formula (1-1) or (1-2); when there is more than one of pdz, they may be the same or different; Y¹ represents —C(R^{a})(R^{b})—, —C(═O)—, —N(R^{c})—, —O—, —Si(R^{d})(R^{e})—, —P(R^{f})—, —S—, or —S(═O)₂; n is an integer of 0 to 5; Ar¹ represents an aryl group that may have a substituent or a monovalent heterocyclic group that may have a substituent; when there is more than one Y¹, they may be the same or different; and R^{a}, R^{b}, R^{c}, R^{d}, R^{e} and R^{f} each independently represent a hydrogen atom or a monovalent substituent, and a compound having a residue thereof are mentioned. Note that the type of pyridazine ring structure present in a single molecule is at least single.

Examples of the aryl group represented by Ar¹ include a phenyl group, a C₁ to C₁₂ alkoxyphenyl group (“C₁ to C₁₂ alkoxy” means that the alkoxy moiety has 1 to 12 carbon atoms. The same applies hereinafter), a C₁ to C₁₂ alkylphenyl group (“C₁ to C₁₂ alkyl” means that the alkyl moiety has 1 to 12 carbon atoms. The same applies hereinafter.), a 1-naphthyl group, a 2-naphthyl group and a pentafluorophenyl group, and preferably a phenyl group, a C₁ to C₁₂ alkoxyphenyl group and a C₁ to C₁₂ alkylphenyl group.

The monovalent heterocyclic group represented by Ar¹ refers to the remaining atomic group provided by removing a single hydrogen atom from a heterocyclic compound. The heterocyclic compound herein refers to an organic compound having a cyclic structure whose constituent elements are not only a carbon atom but also a hetero atom such as an oxygen atom, a sulfur atom, a nitrogen atom and a phosphor atom.

Examples of the monovalent substituents represented by R^{a}, R^{b}, R^{c}, R^{d}, R^{e} and R^{f} include an alkyl group, an alkoxy group, an alkylthio group, an aryl group, an aryloxy group, an arylthio group, an arylalkyl group, an arylalkoxy group, an arylalkylthio group, an arylalkenyl group, an arylalkynyl group an amino group, a substituted amino group, a silyl group, a substituted silyl group, a silyloxy group, a substituted silyloxy group, a monovalent heterocyclic group and a halogen atom.

Note that the compound having a pyridazine ring structure preferably has a structure except a residue of a compound represented by the following general formula (3-3):

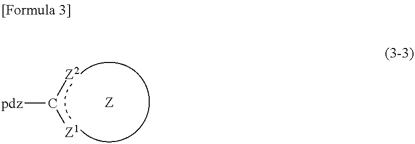

wherein, pdz is as defined above. Ring Z is a cyclic structure containing carbon atoms, Z¹ and Z²; and Z¹ and Z² each independently represent —C(H)═ or —N═.

In the above formula (3-3), as the cyclic structure, an aromatic ring that may have a substituent and a non-aromatic ring that may have a substituent are mentioned. A benzene ring, a heterocyclic ring, an alicyclic hydrocarbon ring, a ring formed by condensing these rings and these rings whose hydrogen atoms are partly substituted are preferable.

The residues of compounds represented by the above formulas (3-1) to (3-3) each refer to a group provided by removing all or some of the hydrogen atoms of the compound.

The compound having a pyridazine ring structure may contain another partial structure. An another preferable partial structure differs depending upon whether it is present at an end or not.

When another partial structure is present at an end, any substituent may be accepted as long as it is stable. Monovalent substituents represented by the above R and R¹ and a hydrogen atom are preferable in view of easiness of synthesis.

When another partial structure is not present at an end, any polyvalent group may be accepted as long as it is stable, and a polyvalent group having a conjugating property is preferable in view of an LUMO energy level. Specific examples of such a group include a divalent aromatic group and a trivalent aromatic group. The aromatic group herein refers to a group derived from an aromatic organic compound. Examples of such an aromatic group include groups provided by replacing n′ (n′ is 2 or 3) hydrogen atoms of an aromatic ring, such as benzene, naphthalene, anthracene, pyridine, quinoline and isoquinoline, by bonds.

Examples of another preferable partial structure that may be included in the compound having a pyridazine ring structure include a structure represented by the following formula (4):

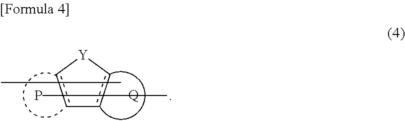

The structure represented by the above formula (4) may have a substituent selected from the group consisting of an alkyl group, an alkoxy group, an alkylthio group, an aryl group, an aryloxy group, an arylthio group, an arylalkyl group, an arylalkoxy group, an arylalkylthio group, an arylalkenyl group, an arylalkynyl group, an amino group, a substituted amino group, a silyl group, a substituted silyl group, a halogen atom, an acyl group, an acyloxy group, an imine residue, an amide group, an acid imide group, a monovalent heterocyclic group, a carboxyl group, a substituted carboxyl group and a cyano group.

In the above formula (4), ring P and ring Q each independently represent an aromatic ring; however, ring P may exist or not. When ring P is present, two bonds are present one on ring P and one on ring Q. When ring P is not present, two bonds are present one on a 5-membered ring or 6-membered ring including Y and one on ring Q. Furthermore, on ring P, ring Q and a 5-membered ring or 6-membered ring including Y, a substituent may be present, which is selected from the group consisting of an alkyl group, an alkoxy group, an alkylthio group, an aryl group, an alkenyl group, an alkynyl group, an aryloxy group, an arylthio group, an arylalkyl group, an arylalkoxy group, an arylalkylthio group, an arylalkenyl group, an arylalkynyl group, an amino group, a substituted amino group, a silyl group, a substituted silyl group, a halogen atom, an acyl group, an acyloxy group, an imine residue, an amide group, an acid imide group, a monovalent heterocyclic group, a carboxyl group, a substituted carboxyl group and a cyano group. As the substituent, a substituent selected from the group consisting of an alkyl group, an alkoxy group, an alkylthio group, an aryl group, an aryloxy group, an arylthio group, an arylalkyl group, an arylalkoxy group, an arylalkylthio group, an arylalkenyl group, an arylalkynyl group, an amino group, a substituted amino group, a silyl group, a substituted silyl group, a halogen atom, an acyl group, an acyloxy group, an imine residue, an amide group, an acid imide group, a monovalent heterocyclic group, a carboxyl group, a substituted carboxyl group and a cyano group is preferable. Y represents —O—, —S—, —Se—, —B(R⁰)—, —Si(R²)(R³)—, P(R⁴)—, P(R⁵)(═O)—, C(R⁶)(R⁷)—, —N(R⁸)—, —C(R⁹)(R¹⁰)—C(R¹¹)(R¹²)—, —O—C(R¹³)(R¹⁴)—, —S—C(R¹⁵)(R¹⁶)—, —N—C(R¹⁷)(R¹⁸)—, —Si(R¹⁹)(R²⁰)—C(R²¹)(R²²)—, —Si(R²³)(R²⁴)—Si(R²⁵)(R²⁶)—, —C(R²⁷)═C(R²⁸)—, —N═C(R²⁹)—, or —Si(R³⁰)═C(R³¹)—. R⁰, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰ and R³¹ herein each independently represent a hydrogen atom, an alkyl group, an alkoxy group, an alkylthio group, an aryl group, an alkenyl group, an alkynyl group, an aryloxy group, an arylthio group, an arylalkyl group, an arylalkoxy group, an arylalkylthio group, an arylalkenyl group, an arylalkynyl group, an amino group, a substituted amino group, a silyl group, a substituted silyl group, a silyloxy group, a substituted silyloxy group, a monovalent heterocyclic group or a halogen atom. Of them, a hydrogen atom, an alkyl group, an alkoxy group, an alkylthio group, an aryl group, an aryloxy group, an arylthio group, an arylalkyl group, an arylalkoxy group, an arylalkylthio group, an arylalkenyl group, an arylalkenyl group, an amino group, a substituted amino group, a silyl group, a substituted silyl group, a silyloxy group, a substituted silyloxy group, a monovalent heterocyclic group and a halogen atom are preferable; an alkyl group, an alkoxy group, an alkylthio group, an aryl group, an aryloxy group, an arylthio group, an arylalkyl group, an arylalkoxy group and a monovalent heterocyclic group are more preferable; an alkyl group, an alkoxy group, an aryl group and a monovalent heterocyclic group are further preferable; and an alkyl group and an aryl group are particularly preferable.

Examples of the structure represented by the above formula (4) include a structure represented by the following formula (4-1), (4-2) or (4-3):

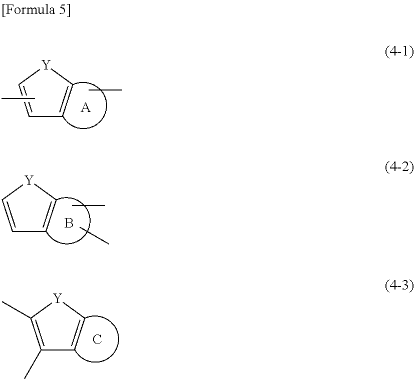

wherein, ring A, ring B and ring C each independently represent an aromatic ring; formulas (4-1), (4-2) and (4-3) each may have a substituent selected from the group consisting of an alkyl group, an alkoxy group, an alkylthio group, an aryl group, an aryloxy group, an arylthio group, an arylalkyl group, an arylalkoxy group, an arylalkylthio group, an arylalkenyl group, an arylalkynyl group, an amino group, a substituted amino group, a silyl group, a substituted silyl group, a halogen atom, an acyl group, an acyloxy group, an imine residue, an amide group, an acid imide group, a monovalent heterocyclic group, a carboxyl group, a substituted carboxyl group and a cyano group; and Y is as defined above, and a structure represented by the following formula (4-4) or (4-5):

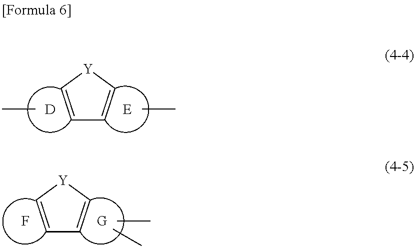

wherein, ring D, ring E, ring F and ring G each independently represent an aromatic ring that may have a substituent selected from the group consisting of an alkyl group, an alkoxy group, an alkylthio group, an aryl group, an aryloxy group, an arylthio group, an arylalkyl group, an arylalkoxy group, an arylalkylthio group, an arylalkenyl group an arylalkynyl group, an amino group, a substituted amino group, a silyl group, a substituted silyl group, a halogen atom, an acyl group, an acyloxy group, an imine residue, an amide group, an acid imide group, a monovalent heterocyclic group, a carboxyl group, a substituted carboxyl group and a cyano group; and Y is as defined above. In the formulas (4-4) and (4-5), Y is preferably a carbon atom, a nitrogen atom, an oxygen atom or a sulfur atom in view of obtaining a high luminous efficiency.

In the formulas (4-1), (4-2), (4-3), (4-4) and (4-5), examples of aromatic rings represented by ring A, ring B, ring C, ring D, ring E, ring F and ring G and having no substituents include aromatic hydrocarbon rings such as a benzene ring, a naphthalene ring, an anthracene ring, a tetracene ring, a pentacene ring, a pyrene ring and a phenanthrene ring; and heteroaromatic rings such as a pyridine ring, a bipyridine ring, a phenanthroline ring, a quinoline ring, an isoquinoline ring, a thiophene ring, a furan ring and a pyrrole ring. These aromatic rings may have the aforementioned substituents.

Furthermore, examples of another preferable partial structure that may be contained in the compound having a pyridazine ring structure include an aromatic amine structure represented by the following formula:

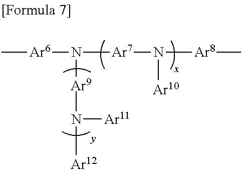

wherein, Ar⁶, Ar⁷, Ar⁸ and Ar⁹ each independently represent an arylene group or a divalent heterocyclic group; Ar¹⁰, Ar¹¹ and Ar¹² each independently represent an aryl group or a monovalent heterocyclic group; Ar⁶, Ar⁷, Ar⁸, Ar⁹, Ar¹⁰, Ar¹¹ and Ar¹² may have a substituent; and x and y each independently represent 0 or 1 and satisfy 0≦x+y≦1.

The arylene group represented by each of Ar⁶, Ar⁷, Ar⁸ and Ar⁹ is the remaining atomic group provided by removing two hydrogen atoms from an aromatic hydrocarbon. Examples of the aromatic hydrocarbon include a compound having a condensed ring and a compound having at least two independent benzene rings or condensed rings directly bonded or bonded via e.g., a vinylene group.

A divalent heterocyclic group represented by each of Ar⁶, Ar⁷, Ar⁸ and Ar⁹ is the remaining atomic group provided by removing two hydrogen atoms from a heterocyclic compound. The number of carbon atoms of the divalent heterocyclic group is usually around 4 to 60. The heterocyclic compound refers to an organic compound having a cyclic structure and containing not only carbon atoms but also hetero atoms such as oxygen, sulfur, nitrogen, phosphorus, boron as elements constituting the ring. As the divalent heterocyclic group, a divalent aromatic heterocyclic group is preferable.

An aryl group represented by each of Ar¹⁰, Ar¹¹ and Ar¹² is the remaining atomic group provided by removing a single hydrogen atom from an aromatic hydrocarbon. The aromatic hydrocarbon is as defined above.

A monovalent heterocyclic group represented by each Ar¹⁰, Ar¹¹ and A₁₂ refers to as the remaining atomic group provided by removing a single hydrogen atom from a heterocyclic compound. The number of carbon atoms of the monovalent heterocyclic group is usually around 4 to 60. The heterocyclic compound is as defined above. As the monovalent heterocyclic group, a monovalent aromatic heterocyclic group is preferable.

When the compound having a pyridazine ring structure is a macromolecular compound, the polystyrene equivalent weight average molecular weight of the compound is preferably 3×10² or more in view of film formation property, more preferably, 3×10² to 1×10⁷, further preferably, 1×10³ to 1×10⁷, and particularly preferably, 1×10⁴ to 1×10⁷.

The compound having a pyridazine ring structure can be used in a wide emission wavelength region. For this, the value of the lowest triplet excitation energy (hereinafter referred to also as “T₁ energy”) of the compound is preferably 2.7 eV or more, more preferably 2.8 eV or more, further preferably 3.0 eV or more, and particularly preferably, 3.1 eV or more. Furthermore, the upper limit is usually 3.5 eV.

The absolute value of the LUMO energy level of the compound having a pyridazine ring structure is preferably 1.5 eV or more, more preferably, 1.6 eV or more, further preferably 1.8 eV or more, and particularly preferably 2.0 eV or more. Furthermore, the upper limit is usually 3.5 eV.

In the specification, a T₁ energy value of each compound and a value of an LUMO energy level are the values calculated by a computational scientific approach. In the specification, as the computational scientific approach, optimization of a ground state structure was performed by the Hartree-Fock (HF) method using a quantum chemical calculation program, Gaussian03, and then, in the optimized structure, a T₁ energy value and a value of an LUMO energy level were obtained by using a B3P86 level time-dependent density functional method. At this time, as a basis function, 6-31g* was used.

In the case where the compound having a pyridazine ring structure is constituted of single-type repeating units, assuming that the unit is represented by A, the compound having a pyridazine ring structure is expressed by the following formula:

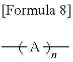

wherein, n represents the number of polymerization units. Herein, a T₁ energy value and a value of an LUMO energy level are calculated in the cases of structures given by n=1, 2 and 3. The T₁ energy value and the value of an LUMO energy level calculated are linearly approximated as a function of (1/n). The values of n=∞ of this case are defined as the T₁ energy value and the value of the LUMO energy level of the macromolecular compound.

In the case where there is more than one repeating unit for constituting the compound having a pyridazine ring structure, T₁ energy values for all cases assuming that n=∞ (wherein n is the number of repeating units polymerized) are calculated in the same manner as described above. Of them, the lowest T₁ energy value is defined as the T₁ energy value of the compound. The value of the LUMO energy level of the macromolecular compound is defined as a value at n=∞ in the repeating unit providing the lowest T₁ energy value. In the present invention, the absolute value of the “value of an LUMO energy level” (more specifically, in the case where the value of an LUMO energy level is expressed by a negative value, the absolute value refers to the value provided by eliminating the negative symbol from the negative value) is important.

When the compound having a pyridazine ring structure contains a pyridazine ring structure represented by the above general formula (1-1), (1-2), (2-1), (2-2), (2-3) or (2-4), a partial structure is preferably present in adjacent to the pyridazine ring structure and the partial structure has at least two it-conjugated electrons. The dihedral angle between the pyridazine ring structure represented by the above general formula (1-1), (1-2), (2-1), (2-2), (2-3) or (2-4) and a partial structure (the partial structure has at least two π-conjugated electrons) adjacent to the pyridazine ring structure is preferably 20° or more, more preferably 30° or more, further preferably 40° or more, especially preferably 50° or more, and particularly preferably, 60° or more.

Furthermore, in the compound having a pyridazine ring structure, dihedral angles between aromatic rings and hetero aromatic rings including the pyridazine ring structure all are preferably 20° or more, more preferably 40° or more, further preferably 50° or more, and particularly preferably 60° or more. Furthermore, to obtain such a dihedral angle, it is preferable not to have a pyridazine ring structure represented by the above general formula (3-3).

Here, in the specification, the dihedral angle refers to an angle calculated from the optimized structure in a normal state. The dihedral angle is defined, for example, by a carbon atom (a₁) which is located at a bonding position and the carbon atom or nitrogen atom (a₂) located next to a₁ in the compound having a pyridazine ring structure represented by the general formula (1-1), (1-2), (2-1), (2-2), (2-3) or (2-4), and an atom (a₃) located in the binding position and an atom (a₄) located next to a₃ in a structure binding to the pyridazine ring structure. If more than one atom (a₂) or atom (a₄) can be selected herein, dihedral angles of all cases are calculated. Of them, the value having the lowest absolute value is employed as the dihedral angle. The atom (a₃) and the atom (a₄) are atoms having it-conjugated electrons, and more preferably, are carbon atoms, nitrogen atoms, silicon atoms and phosphorus atoms. In the specification, calculation is made from an optimized structure (more specifically, the structure produced with the lowest formation energy) at n=3 (n is the number of polymerization units) in a normal state obtained by a computational scientific approach. In the compound having a pyridazine ring structure, when there are a plurality of pyridazine ring structures, there is more than one dihedral angle. In this case, all dihedral angles of the macromolecular compound preferably satisfy the above conditions.

As the compound having a pyridazine ring structure, compounds represented by the following formulas (5-1) to (5-22) are mentioned. In the following formulas (5-1) to (5-22), R* represents a hydrogen atom or a monovalent substituent. Examples of the monovalent substituent represented by R* include a halogen atom, an alkyl group, an alkoxy group, an alkylthio group, an aryl group that may have a substituent, an aryloxy group, an arylthio group, an arylalkyl group, an arylalkyloxy group, an arylalkylthio group, an acyl group, an acyloxy group, an amide group, an acid imide group, an imide residue, a substituted amino group, a substituted silyl group, a substituted silyloxy group, a substituted silylthio group, a substituted silylamino group, a monovalent heterocyclic group that may have a substituent, an heteroaryl group that may have a substituent, a heteroaryloxy group, a heteroarylthio group, an arylalkenyl group, an arylethynyl group, a substituted carboxyl group and a cyano group. More than one R* may be the same or different. As R*, an alkyl group, an alkoxy group, an aryl group that may have a substituent and a heteroaryl group that may have a substituent are more preferable. More than one R* may be the same or different.

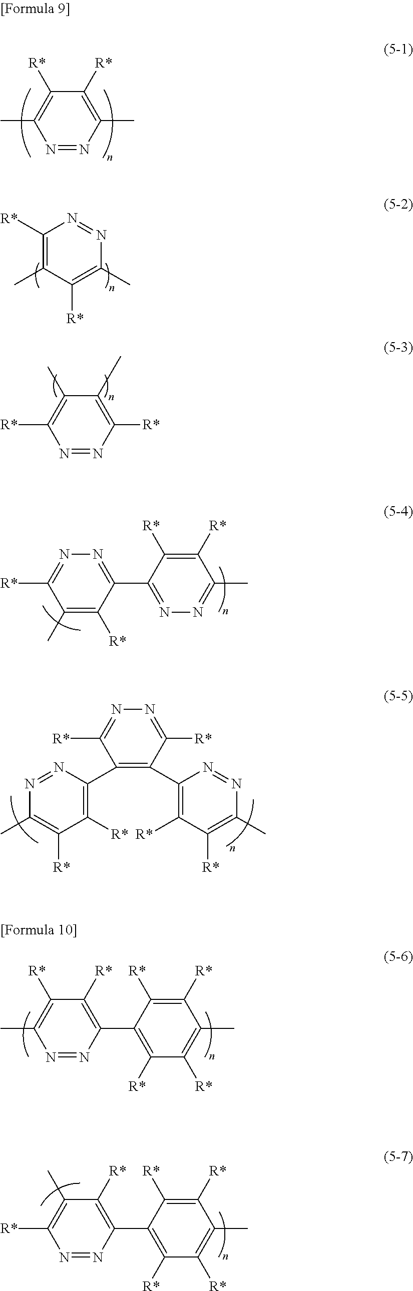

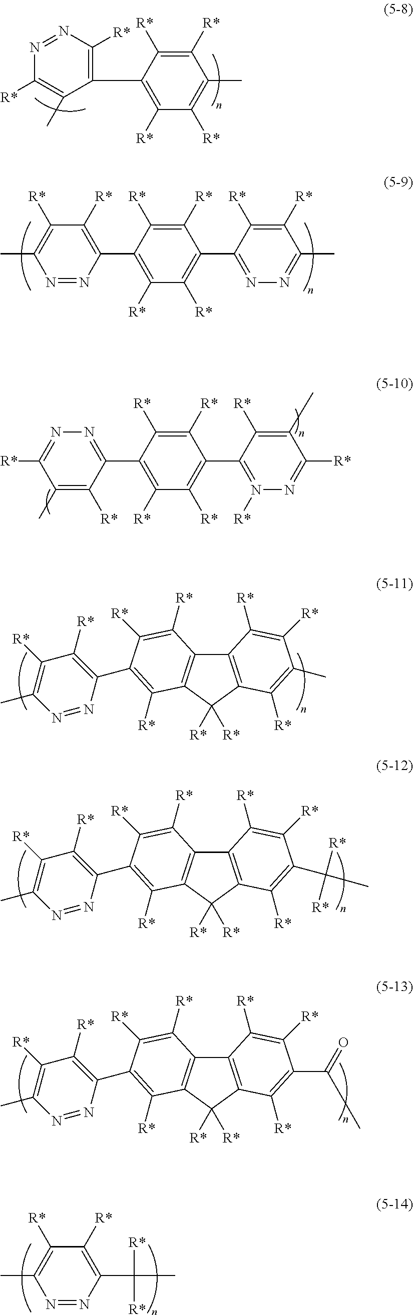

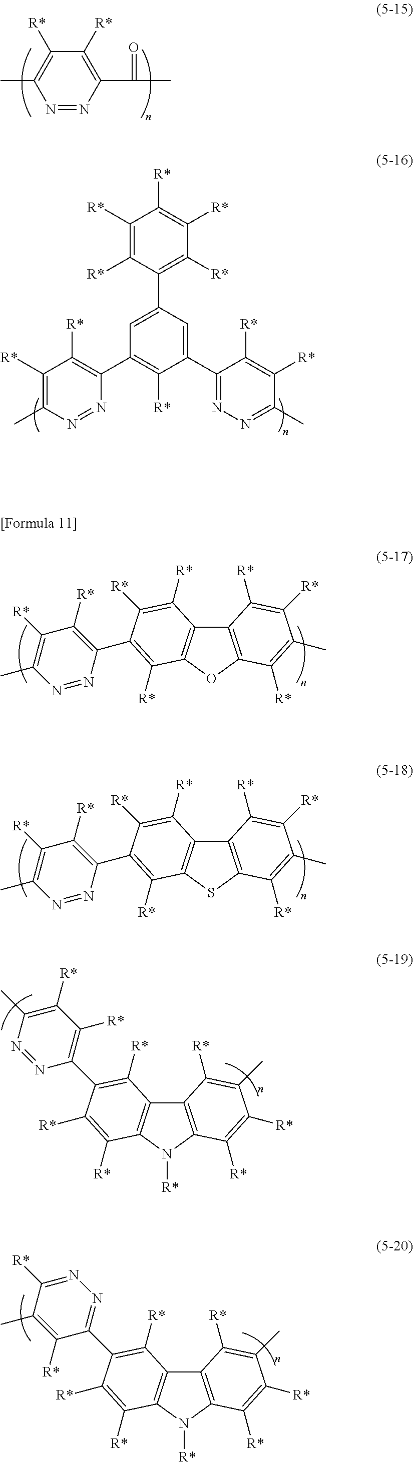

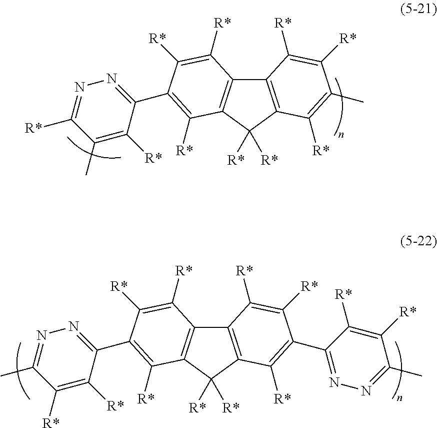

wherein, n represents the number of polymerization units.

Furthermore, as the compound having a pyridazine ring structure, the following compounds may also be mentioned.

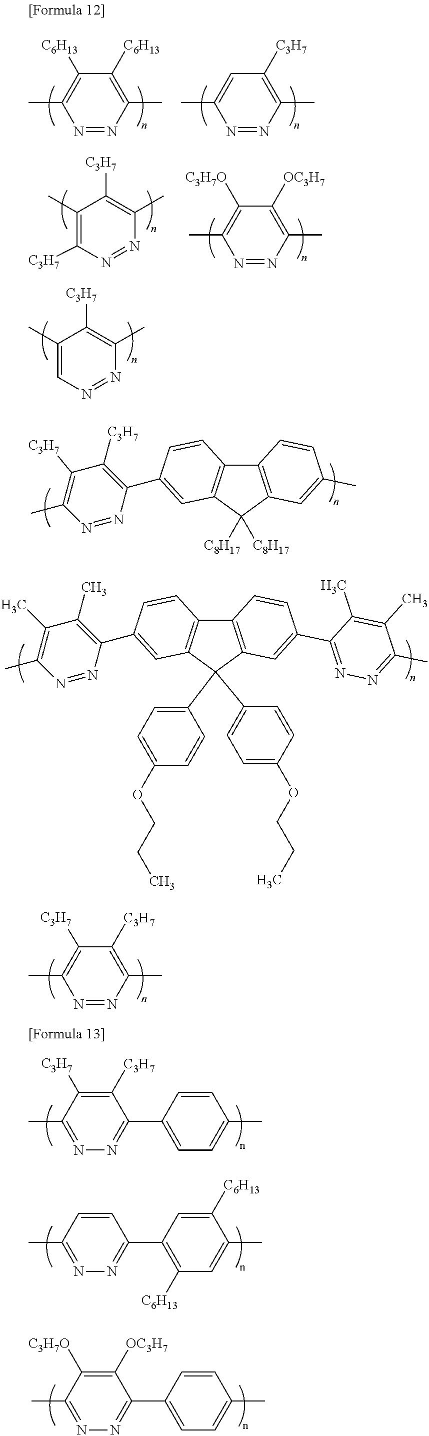

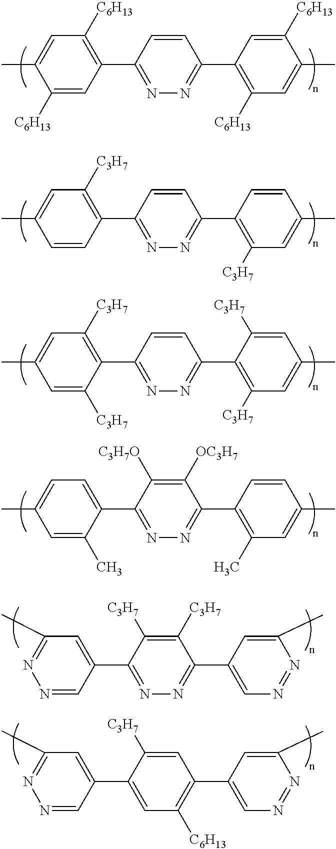

wherein, n represents the number of polymerization units.

Furthermore, as the compound having a pyridazine ring structure, the following compounds may also be mentioned.

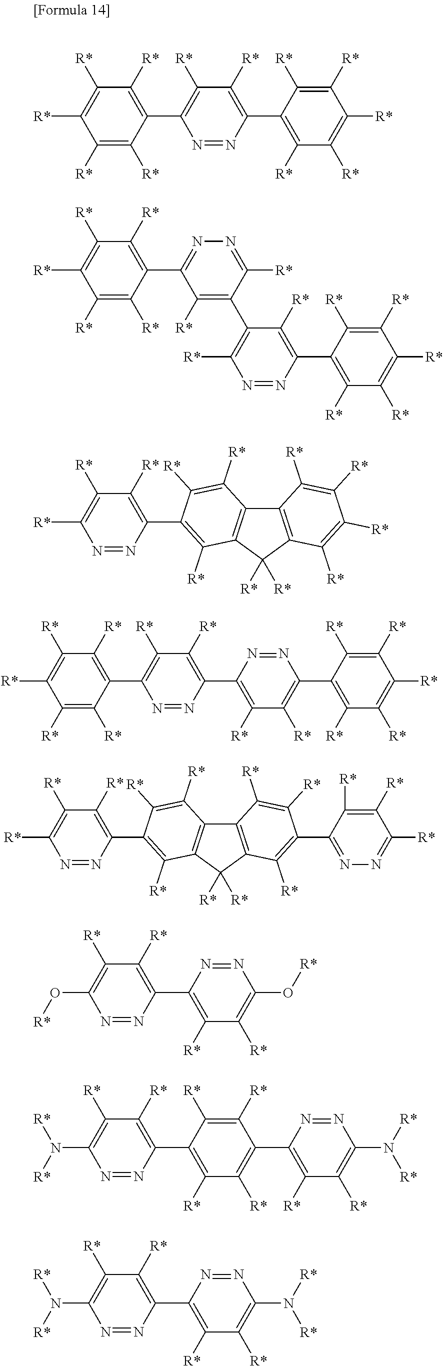

Furthermore, as the compound having a pyridazine ring structure, the following compounds may also be mentioned.

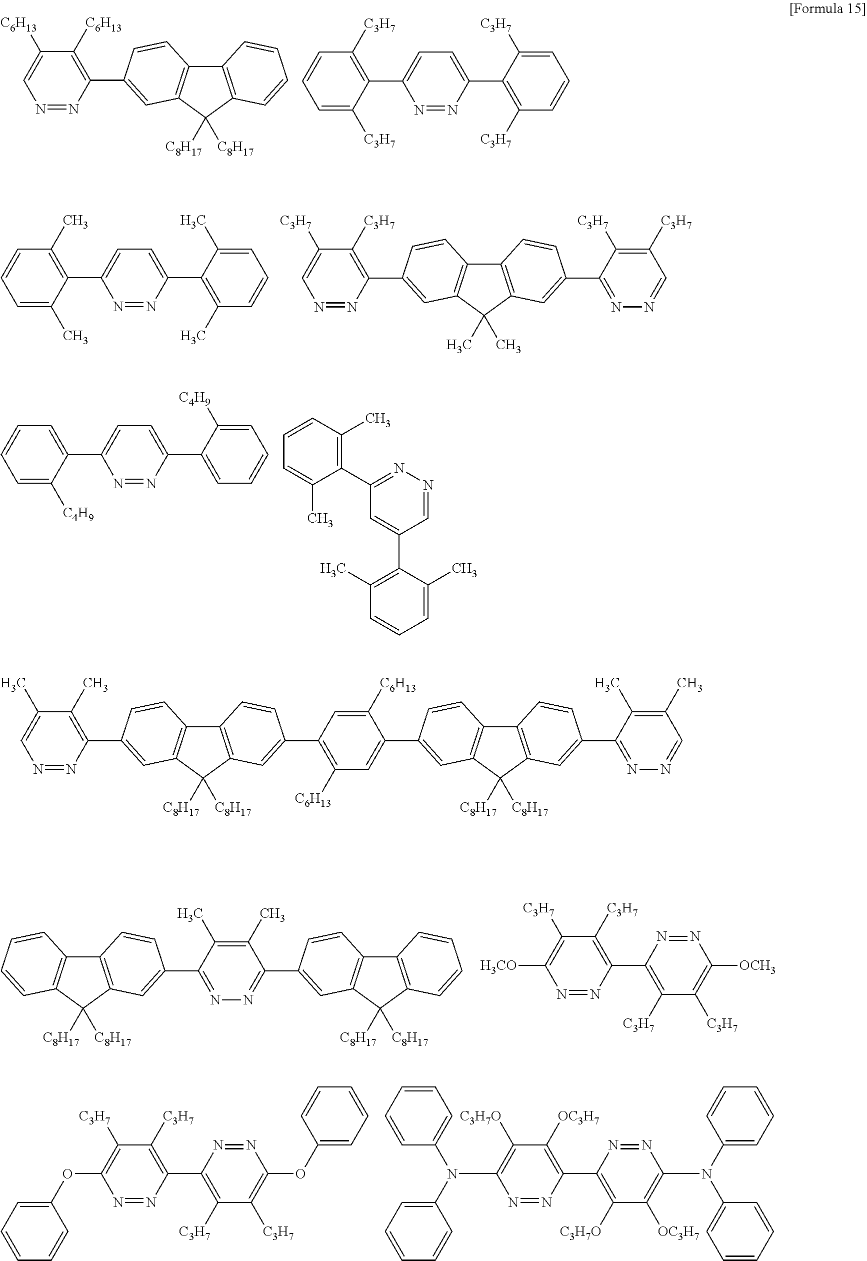

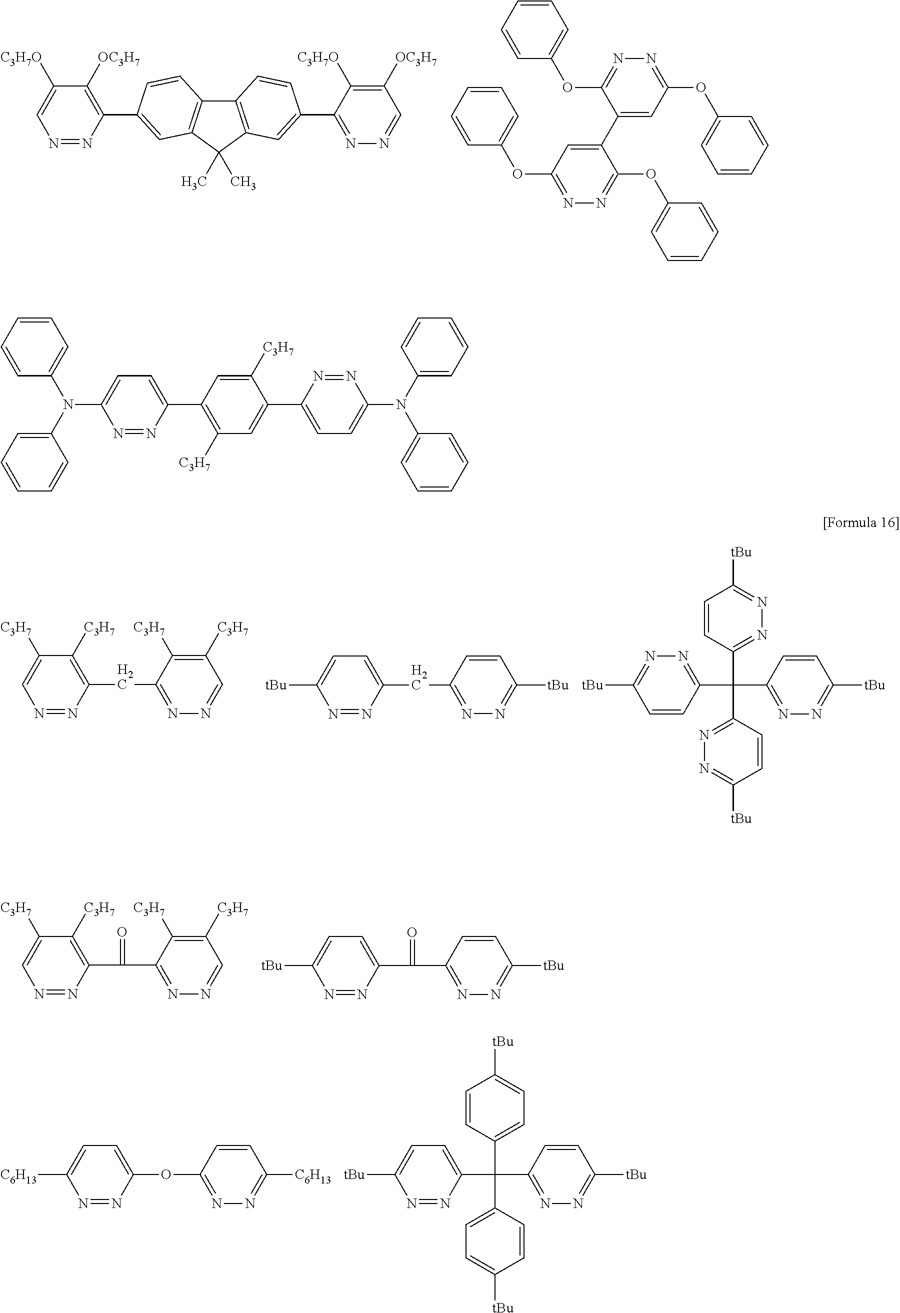

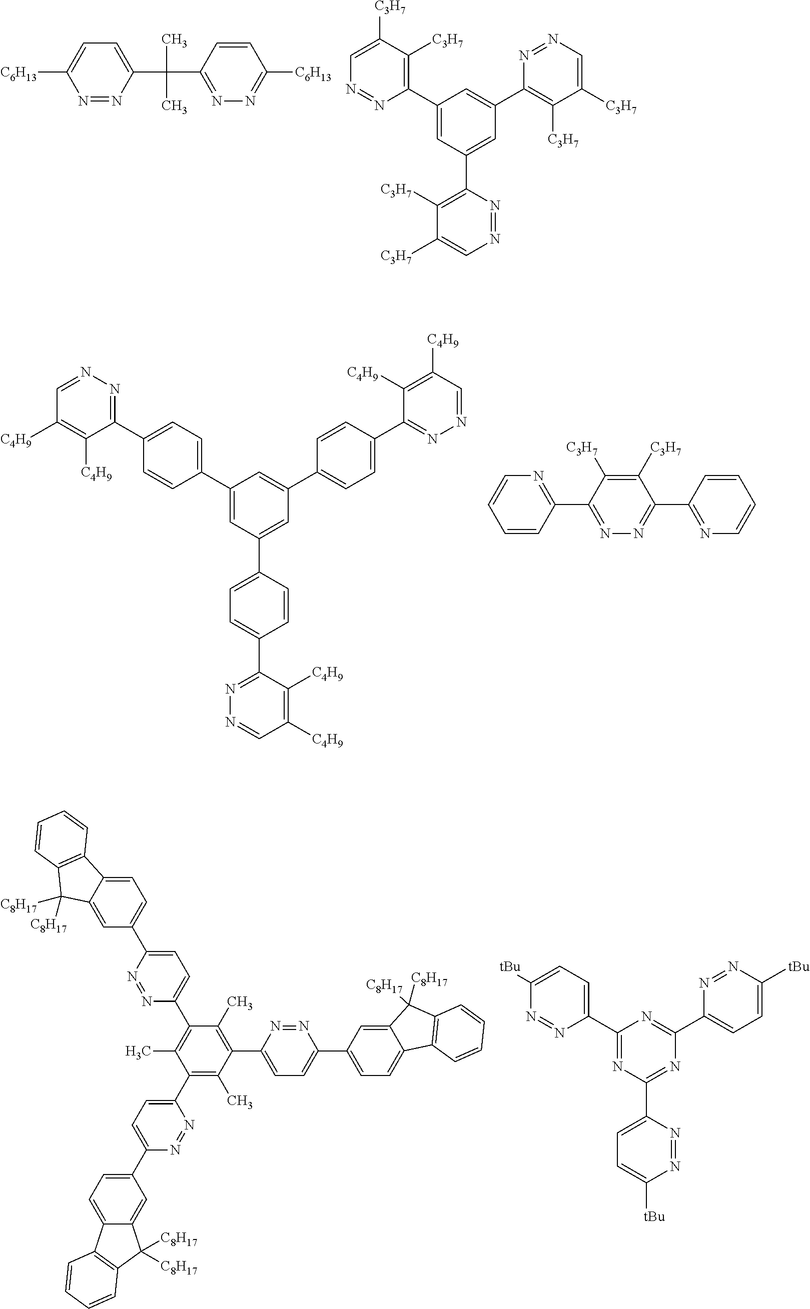

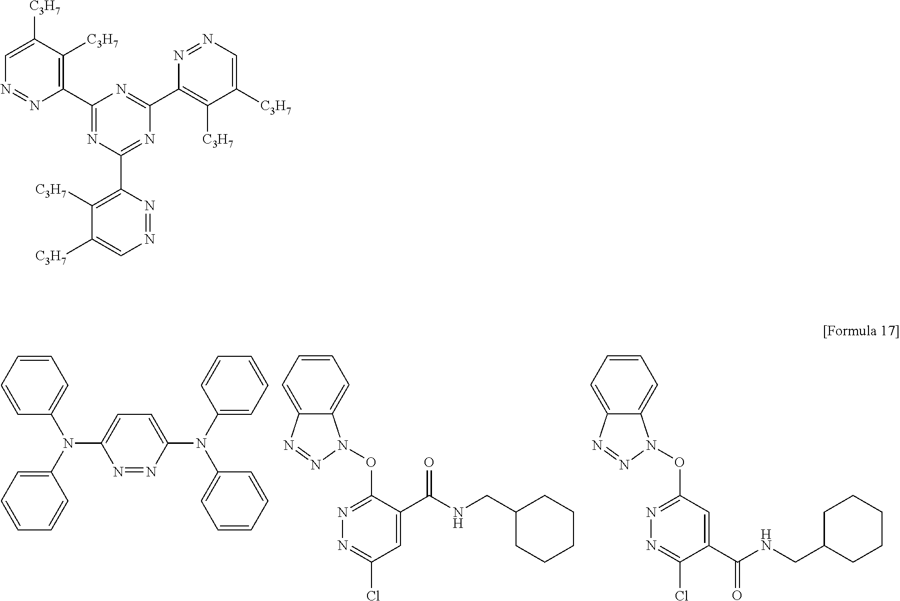

As the phosphorescent compound, known compounds such as triplet emission complexes can be used. For example, compounds that have conventionally been used as a lower-molecular EL emitting material are mentioned. They are disclosed, for example, in Nature, (1998), 395, 151, Appl. Phys. Lett. (1999), 75(1), 4, Proc. SPIE-Int. Soc. Opt. Eng. (2001), 4105 (Organic Light-Emitting Materials and Devices IV), 119, J. Am. Chem. Soc., (2001), 123, 4304, Appl. Phys. Lett., (1997), 71(18), 2596, Syn. Met., (1998), 94(1), 103, Syn. Met., (1999), 99(2), 1361, Adv. Mater., (1999), 11(10), 852, Inorg. Chem., (2003), 42, 8609, Inorg. Chem., (2004), 43, 6513, Journal of the SID 11/1, 161 (2003), WO2002/066552, WO2004/020504, and WO2004/020448. Of them, the total of a square of an orbital coefficient of the outermost shell d-orbital of the central metal in the HOMO of a metal complex preferably occupies not less than 1/3 ratio of the total of a square of orbital coefficients of all atoms in order to obtain a high luminous efficiency. For example, ortho-metalated complexes, which is a transition metal having a central metal belonging to the 6th period, are mentioned.

The central metal of the triplet emission complex, which is usually a metal atom of an atomic number of 50 or more, having a spin-orbit interaction, and capable of causing the intersystem crossing between a singlet state and a triplet state, includes preferably atoms such as gold, platinum, iridium, osmium, rhenium, tungsten, europium, terbium, thulium, dysprosium, samarium, praseodymium, gadolinium and ytterbium; more preferably atoms such as gold, platinum, iridium, rhenium and tungsten; further preferably atoms such as gold, platinum, iridium, osmium and rhenium; and particularly preferably atoms such as platinum and iridium.

Examples of the ligand of the triplet emission complex include 8-quinolinol and a derivative thereof, benzoquinolinol and a derivative thereof, and 2-phenyl-pyridine and a derivative thereof.

As the phosphorescent compound, in view of solubility, a compound having a substituent such as an alkyl group, an alkoxy group, an aryl group that may have a substituent and a heteroaryl group that may have a substituent are preferable. Furthermore, the substituent preferably has 3 or more atoms in total, except a hydrogen atom, more preferably 5 or more, further preferably 7 or more, and particularly preferably 10 or more. Furthermore, at least one of the substituents is preferably present in each ligand. The types of substituents may be the same or different per ligand.

As the phosphorescent compound, the following compounds are mentioned.

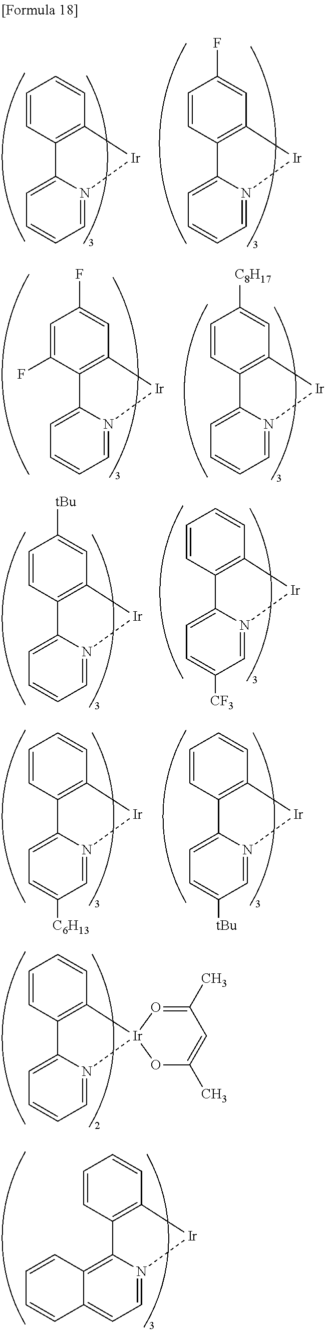

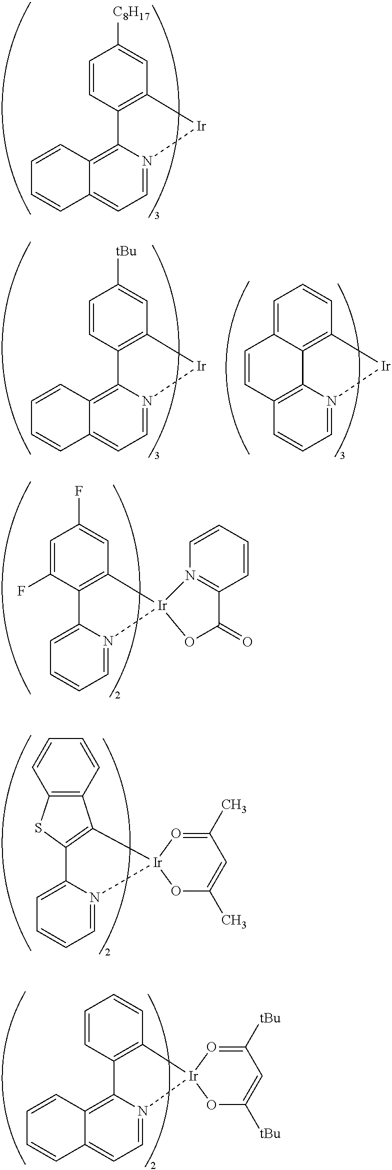

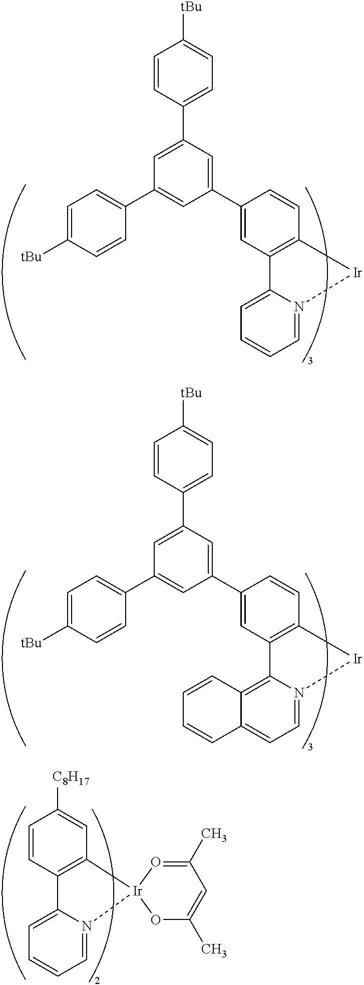

The amount of phosphorescent compound in the composition of the present invention varies depending upon the type of organic compound to be used in combination and the properties to be optimized and is therefore not particularly limited; however, the amount is usually, 0.01 to 80 parts by weight, based on 100 parts by weight of the compound having a pyridazine ring structure, preferably, 0.1 to 30 parts by weight, more preferably, 0.1 to 15 parts by weight, and particularly preferably, 0.1 to 10 parts by weight. Note that in the composition of the present invention, the compound having a pyridazine ring structure and the phosphorescent compound may each be used alone or in combination of two or more thereof.

The composition of the present invention may contain an optional component other than the compound having the pyridazine ring structure and the phosphorescent compound as long as the object of the invention is not damaged. As the optional component, for example, a hole transport material, an electron transport material and an antioxidant are mentioned.

Examples of the hole transport material include well-known hole transport materials for an organic EL device, such as an aromatic amine, a carbazole derivative and a polyparaphenylene derivative.

Examples of the electron transport material include well-known electron transport materials for an organic EL device, such as metal complexes of an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, and 8-hydroxyquinoline and a derivative thereof.

In the composition of the present invention, the value of the lowest triplet excitation energy (ETP) of the compound having a pyridazine ring structure and the value of the lowest triplet excitation energy (ETT) of the phosphorescent compound preferably satisfy the following expression:

ETP>ETT−0.2 (eV)

in view of highly efficient light emission, more preferably satisfy

ETP>ETT (eV)

and further preferably

ETP>ETT+0.1 (eV)

and particularly preferably

ETP>ETT+0.2 (eV).

The light-emitting thin film of the present invention can be obtained by using forming a thin film consisting of the composition, etc. of the present invention. For preparing the thin film, known methods can be selected and put in use, and solution coating, vapor deposition and transfer, etc. can be used. As the solution coating, a spin coating method, a casting method, a microgravure coating method, a gravure coating method, a bar coating method, a roll coating method, a wire-bar coating method, dip coating method, a spray coating method, a screen printing method, a flexo printing method, an off-set printing method and an inkjet printing method etc. may be used.

As the solvent, a solvent capable of dissolving or uniformly dispersing the composition is preferable. Examples of the solvent include chlorine solvents (chloroform, methylene chloride, 1,2-dichloroethane, 1,1,2-trichloroethane, chlorobenzene, o-dichlorobenzene, etc.), ether solvents (tetrahydrofuran, dioxane, etc.), aromatic hydrocarbon solvents (toluene, xylene, etc.), aliphatic hydrocarbon solvents (cyclohexane, methylcyclohexane, n-pentane, n-hexane, n-heptane, n-octane, n-nonane, n-decane, etc.), ketone solvents (acetone, methyl ethyl ketone, cyclohexanone, etc.), ester solvents (ethyl acetate, butyl acetate, ethyl cellosolve acetate, etc.), polyhydric alcohols and derivatives thereof (ethylene glycol, ethylene glycol monobutyl ether, ethylene glycol monoethyl ether, ethylene glycol monomethyl ether, dimethoxyethane, propylene glycol, diethoxymethane, triethylene glycol monoethyl ether, glycerin, 1,2-hexanediol, etc.), alcohol solvents (methanol, ethanol, propanol, isopropanol, cyclohexanol, etc.), sulfoxide solvents (dimethylsulfoxide, etc.) and amide solvents (N-methyl-2-pyrrolidone, N,N-dimethylformamide, etc.). A solvent can be selected from them and put in use. Furthermore, these organic solvents may be used alone or in combination of two or more thereof.

When the inkjet printing method is used, to improve ejection property from a head and uniformity, etc., a solvent in a solution and additives can be selected according to known methods. In this case, the viscosity of the solution is preferably 1 to 100 mPa·s at 25° C. Furthermore, if vaporization is significant, it tends to be difficult to repeat ejection from a head. In view of them, examples of a solvent used include a single solvent or solvent mixture containing anisole, bicyclohexyl, xylene, tetralin and dodecyl benzene. Generally, a solution for inkjet printing suitable for a composition to be used can be obtained by a method of mixing more than one solvent, a method of controlling the concentration thereof in a solution of a composition and the like.

### <Macromolecular Compound>

The macromolecular compound of the present invention is a macromolecular compound having a residue of a phosphorescent compound and a pyridazine ring structure. The phosphorescent compound and the pyridazine ring structure are the same exemplified in the above section of composition. Examples of the macromolecular compound of the present invention include (1) a macromolecular compound having a structure of a phosphorescent compound in the main chain, (2) a macromolecular compound having a structure of a phosphorescent compound at an end, and (3) a macromolecular compound having a structure of a phosphorescent compound at a side chain.

### <Light-Emitting Device>

Next, the light-emitting device of the present invention will be described.

The light-emitting device of the present invention is prepared by using the composition, etc. of the present invention. Usually, the composition, etc. of the present invention are contained in at least a site between electrodes consisting of an anode and a cathode. They are preferably contained as a light-emitting layer in the form of the light-emitting thin film. Furthermore, in view of improving performance such as luminous efficiency and durability, one or more known layers having another function may be contained. Examples of such a layer include a charge transport layer (more specifically, hole transport layer, electron transport layer), a charge block layer (more specifically, hole block layer, electron block layer), a charge injection layer (more specifically, hole injection layer, electron injection layer), and a buffer layer. Note that in the light-emitting device of the present invention, the light-emitting layer, charge transport layer, charge block layer, charge injection layer and buffer layer, etc. each may be formed of a single layer or two or more layers.

The light-emitting layer is a layer having a function of emitting light. The hole transport layer is a layer having a function of transporting holes. The electron transport layer is a layer having a function of transporting electrons. The electron transport layer and the hole transport layer are collectively referred to as a charge transport layer. Furthermore, the charge block layer is a layer having a function of confining holes or electrons in the light-emitting layer. The layer for transporting electrons and confining holes is referred to as a hole block layer and a layer for transporting holes and confining electrons is referred to as an electron block layer.

As the buffer layer, a layer provided in adjacent to an anode and containing a conductive polymer compound is mentioned.

As the light-emitting device of the present invention, the following structures a) to q) are mentioned.

a) Anode/light-emitting layer/cathode

b) Anode/hole transport layer/light-emitting layer/cathode

c) Anode/light-emitting layer/electron transport layer/cathode

d) Anode/light-emitting layer /hole block layer/cathode

e) Anode/hole transport layer/light-emitting layer/electron transport layer/cathode

f) Anode/charge injection layer/light-emitting layer/cathode

g) Anode/light-emitting layer/charge injection layer/cathode

h) Anode/charge injection layer/light-emitting layer/charge injection layer/cathode

i) Anode/charge injection layer/hole transport layer/light-emitting layer/cathode

j) Anode/hole transport layer/light-emitting layer/charge injection layer/cathode

k) Anode/charge injection layer/hole transport layer/light-emitting layer/charge injection layer/cathode

l) Anode/charge injection layer/light-emitting layer/electron transport layer/cathode

m) Anode/light-emitting layer/electron transport layer/charge injection layer/cathode

n) Anode/charge injection layer/light-emitting layer/electron transport layer/charge injection layer/cathode

o) Anode/charge injection layer/hole transport layer/light-emitting layer/electron transport layer/cathode

p) Anode/hole transport layer/light-emitting layer/electron transport layer/charge injection layer/cathode

q) Anode/charge injection layer/hole transport layer/light-emitting layer/electron transport layer/charge injection layer/cathode (herein, the symbol “/” means that layers are laminated next to each other. The same will be applied in the following. Note that the light-emitting layer, hole transport layer and electron transport layer may each independently be formed of two or more thereof).

In the case where the light-emitting device of the present invention has a hole transport layer (usually, the hole transport layer contains a hole transport material), known materials are mentioned as the hole transport material. Examples thereof include polymer hole transport materials such as polyvinylcarbazole and a derivative thereof, polysilane and a derivative thereof, polysiloxane derivative having an aromatic amine in a side chain or the main chain, a pyrazoline derivative, an arylamine derivative, a stilbene derivative, a triphenyldiamine derivative, polyaniline and a derivative thereof, polythiophene and a derivative thereof, polypyrrole and a derivative thereof, poly(p-phenylenevinylene) and a derivative thereof, and poly(2,5-thienylenevinylene) and a derivative thereof; and further include the compounds described in JP 63-70257 A, JP 63-175860 A, JP 2-135359 A, JP 2-135361 A, JP 2-209988 A, JP 3-37992 A and JP 3-152184 A.

In the case where the light-emitting device of the present invention has an electron transport layer (usually, the electron transport layer contains an electron transport material), known materials are mentioned as the electron transport material. Examples thereof include an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, 8-hydroxyquinoline and a complex of a derivative thereof, polyquinoline and a derivative thereof, polyquinoxaline and a derivative thereof, and polyfluorene and a derivative thereof.

The film thicknesses of the hole transport layer and electron transport layer, whose optimum values thereof vary depending upon the material to be used, may be appropriately selected so as to obtain an appropriate driving voltage and luminous efficiency; however, the thickness is required to be sufficiently thick such that at least pin holes are not formed. If the film is extremely thick, the driving voltage of the device becomes high and thus not preferable. Therefore, the film thicknesses of the hole transport layer and electron transport layer are for example, 1 nm to 1 μm, preferably 2 nm to 500 nm, and further preferably 5 nm to 200 nm.

Furthermore, of the charge transport layers provided in adjacent to an electrode, a charge transport layer having a function of improving a charge injection efficiency from the electrode and an effect of reducing the driving voltage of the device, is sometimes called particularly as a charge injection layer (that is, a general name of a hole injection layer, and an electron injection layer. The same will be applied in the following).

Furthermore, to improve adhesion with an electrode and improve charge injection form an electrode, the charge injection layer or an insulating layer may be provided in adjacent to the electrode (usually, having an average thickness of 0.5 nm to 4 nm. The same will be applied in the following). Furthermore, to improve the adhesion of the interface and prevent contamination, etc., a thin buffer layer may be inserted into the interface of a charge transport layer and a light-emitting layer.

The lamination order of the layers and number of layers and the thickness of individual layers can be appropriately selected in consideration of luminous efficiency and the life of the device.

Examples of the charge injection layer include a layer containing a conductive polymer compound, a layer provided between an anode and a hole transport layer and having an intermediate ionization potential between an anode material and a hole transport material contained in the hole transport layer, and a layer provided between a cathode and an electron transport layer and having an intermediate electron affinity value between a cathode material and an electron transport material contained in the hole transport layer.

The material to be used in the charge injection layer may be appropriately selected in consideration of the materials of electrodes and adjacent layers. Examples thereof include polyaniline and a derivative thereof, polythiophene and a derivative thereof, polypyrrole and a derivative thereof, polyphenylenevinylene and a derivative thereof, polythienylenevinylene and a derivative thereof, polyquinoline and a derivative thereof, polyquinoxaline and a derivative thereof, a conductive polymer compound such as a polymer containing an aromatic amine structure in the main chain or a side chain, a metal phthalocyanine (copper phthalocyanine, etc.) and carbon.

The insulating layer has a function of facilitating charge injection. Examples of the material for the insulating layer include a metal fluoride, a metal oxide and an organic insulating material. As the light-emitting device having the insulating layer provided therein, for example, a light-emitting device having an insulating layer provided in adjacent to a cathode and a light-emitting device having an insulating layer provided in adjacent to an anode are mentioned.

The light-emitting device of the present invention is usually formed on a substrate. Any substrate may be used as long as it does not change even if an electrode is formed thereon and an organic material layer is formed thereon. Examples thereof include glass, plastic, a polymer film and silicon. In the case of an opaque substrate, an opposite electrode is preferably transparent or semitransparent.

At least one of the anode and the cathode present in the light-emitting device of the present invention is usually transparent or semitransparent. Of them, the anode side is preferably transparent or semitransparent.

As a material for an anode, known materials can be appropriately selected and put in use, and usually a conductive metal oxide film and a semitransparent metal thin film, etc. are used. Specific examples thereof include films (NESA, etc.) prepared by using conductive inorganic compounds such as indium oxide, zinc oxide, tin oxide, and a complex thereof, namely, indium tin oxide (ITO), indium zinc oxide; gold, platinum, silver and copper. ITO, indium zinc oxide, and tin oxide are preferable. As the preparation method, a vacuum vapor deposition method, a sputtering method, an ion plating method and a plating method, etc. are mentioned. Furthermore, as the anode, an organic transparent conductive film of polyaniline and a derivative thereof, and polythiophene and a derivative thereof etc. may be used. Note that the anode may be formed of a laminate structure of 2 layers or more.

As a material for a cathode, known materials can be appropriately selected and put in use, and usually, a material having a small work function is preferable. Examples thereof include metals such as lithium, sodium, potassium, rubidium, cesium, beryllium, magnesium, calcium, strontium, barium, aluminum, scandium, vanadium, zinc, yttrium, indium, cerium, samarium, europium, terbium, ytterbium, and alloys formed from at least two of metals selected from them or alloys of at least one of metals selected from them and at least one of gold, silver, platinum, copper, manganese, titanium, cobalt, nickel, tungsten and tin, graphite or a graphite intercalation compound. Examples of the alloy include a magnesium-silver alloy, a magnesium-indium alloy, a magnesium-aluminum alloy, an indium-silver alloy, a lithium-aluminum alloy, a lithium-magnesium alloy, a lithium-indium alloy, a calcium-aluminum alloy. Note that the cathode may be formed of a laminate structure of 2 layers or more.

The light-emitting device of the present invention can be used, for example, as a planar light source, a display (for example, a segment display, a dot matrix display, a liquid crystal display) and backlights thereof (a liquid crystal display having the light-emitting device as a backlight).

To obtain planer emission of light using the light-emitting device of the present invention, a planar anode and cathode are arranged so as to overlap them. Furthermore, to obtain patterned emission of light, there are a method of placing a mask having a patterned window on the surface of the planar light-emitting device, a method of forming an extremely thick organic material layer in a non light-emitting section such that light is not substantially emitted, and a method of forming a patterned electrode as either one of an anode and cathode or both electrodes. Patterns are formed by any one of these methods and electrodes are arranged so as to independently turn ON/OFF. In this manner, a segment-type display device capable of displaying numeric characters and letters, and simple symbols, etc. can be obtained. Furthermore, to obtain a dot matrix device, an anode and a cathode are formed in the form of stripe and arranged so as to cross perpendicularly. A partial color display and multi color display can be provided by a method of distinctively applying more than one light-emitting material different in luminous color and a method of using a color filter or a fluorescence conversion filter. A dot-matrix device can be passively driven or may be actively driven in combination with TFT, etc. These display devices can be used as displays for computers, televisions, mobile terminals, mobile phones, car-navigation and view finders of video cameras, etc.

Furthermore, the planar light-emitting device is usually an autonomous light-emitting thin device and can be preferably used as a planar light source for a backlight of a liquid crystal display and a light (for example, planar lighting, a light source for planar lighting), etc. Furthermore, if a flexible substrate is used, the light-emitting device can be used as a curved-surface light source, a light and a display, etc.

The composition, etc. of the present invention are not only useful for preparing a device but can be also used as a semiconductor material such as an organic semiconductor material, a light-emitting material, an optical material and a conductive material (for example, applied by doping). Accordingly, thin films such as light-emitting thin film, a conductive thin film and an organic semiconductor thin film can be prepared by using the composition, etc. of the present invention.

The composition, etc. of the present invention can be used to form a conductive thin film and a semiconductor thin film in the same manner as in a preparation method for a light-emitting thin film to be used in the light emitting layer of the light-emitting device, and formed into a device. In the semiconductor thin film, a larger value of an electron mobility or hole mobility is preferably not less than 10⁻⁵ cm²/V/second. Furthermore, an organic semiconductor thin film can be used in organic solar batteries and organic transistors, etc.

### EXAMPLES

Hereinafter, Examples will be described to explain the present invention more specifically; however, the present invention is not limited to them.

### Example 1

The value of lowest triplet excitation energy T₁ (1/n=0) of a macromolecular compound (P-1) represented by the following formula:

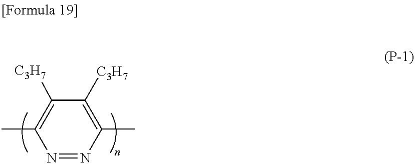

wherein, n is the number of polymerization units,
was 3.0 eV, an absolute value E_{LUMO} of an LUMO energy level (1/n=0) was 1.9 eV, both of which were extrapolation values thereof at n=∞, and the smallest dihedral angle was 67°.

Parameters were calculated by the computational scientific approach. More specifically, using the repeating unit (M-1) of a macromolecular compound (P-1):

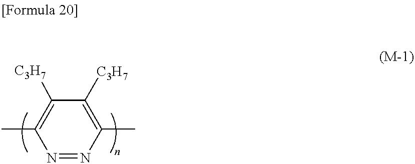

structure optimization was performed by the HF method at n=1, 2 and 3.

At this time, as a basis function, 6-31G* was used. Thereafter, an absolute value of an LUMO energy level and the value of the lowest triplet excitation energy were calculated by a time-dependent density functional method of B3P86 level using the same basis. The absolute value of an LUMO energy level and the value of the lowest triplet excitation energy calculated in each n were each expressed as inverse function (1/n) of n and the extrapolation value at n=∞ was a value of this function at 1/n=0.

Furthermore, dihedral angles were calculated from an optimized structure at n=3 (n is the number of polymerization units). Since more than one pyridazine ring structure is present, there is more than one dihedral angle. Of them, the smallest value alone is described herein.

If a light-emitting device is prepared by using a composition containing a macromolecular compound (P-1) and a phosphorescent compound, it can be confirmed that it has excellent luminous efficiency.

### Example 2

The value of lowest triplet excitation energy T₁ (1/n=0) of a macromolecular compound (P-2) represented by the following formula:

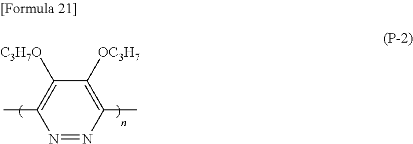

wherein, n is the number of polymerization units,
was 2.9 eV, an absolute value E_{LUMO} of an LUMO energy level (1/n=0) was 2.2 eV, both of which were extrapolation values thereof at n=∞, and the smallest dihedral angle was 59°.

Parameters were calculated in the same manner as in Example 1 by using repeating unit (M-2) of the polymer (P-2):

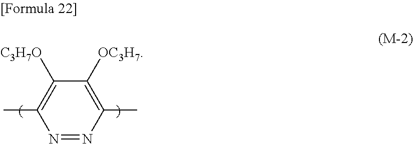

If a light-emitting device is prepared by using a composition containing a macromolecular compound (P-1) and a phosphorescenct compound, it can be confirmed that it has excellent luminous efficiency.

### Example 3

The value of lowest triplet excitation energy T₁ of a compound (C-1) represented by the following formula:

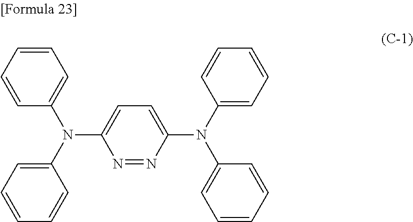

was 2.8 eV, and an absolute value E_{LUMO} of an LUMO energy level was 1.6 eV.

Parameters were calculated by the computational scientific approach. More specifically, structure optimization of the compound (C-1) was performed by the HF method. At this time, as a basis function, 6-31G* was used. Thereafter, an absolute value of an LUMO energy level and the value of the lowest triplet excitation energy were calculated by the time-dependent density functional method of B3P86 level using the same basis.

It can be confirmed that a light-emitting device which is prepared by using a composition containing the compound (C-1) and a phosphorescent compound is excellent in luminous efficiency.

### Example 4

The value of lowest triplet excitation energy T₁ of a compound (C-2) represented by the following formula:

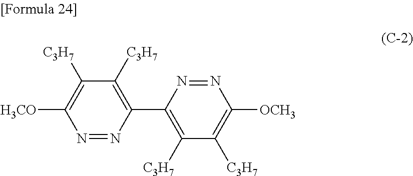

was 3.1 eV and an absolute value E_{LUMO} of an LUMO energy level was 1.6 eV. Note that the value of the lowest triplet excitation energy T₁ and an absolute value of an LUMO energy level were calculated by the computational scientific approach in the same manner as in Example 3.

It can be confirmed that a light-emitting device which is prepared by using a composition containing the compound (C-2) and a phosphorescent compound is excellent in luminous efficiency.

### Example 5

The value of lowest triplet excitation energy T₁ of a compound (C-3) represented by the following formula:

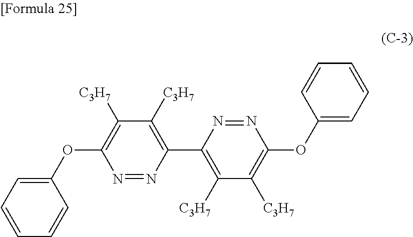

was 3.1 eV and an absolute value E_{LUMO} of an LUMO energy level was 1.7 eV. Note that the value of the lowest triplet excitation energy T₁ and an absolute value of an LUMO energy level were calculated by the computational scientific approach in the same manner as in Example 3.

It can be confirmed that a light-emitting device which is prepared by using a composition containing a compound (C-3) and a phosphorescent compound is excellent in luminous efficiency.

### Example 6

The value of lowest triplet excitation energy T₁ of a compound (C-4) represented by the following formula:

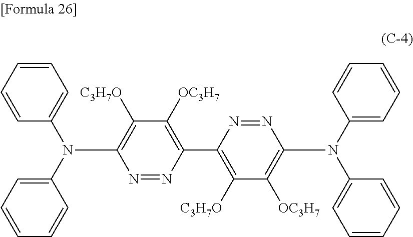

was 3.0 eV and an absolute value of an LUMO energy level E_{LUMO} was 1.7 eV. Note that the value of the lowest triplet excitation energy T₁ and an absolute value of an LUMO energy level were calculated by the computational scientific approach in the same manner as in Example 3.

It can be confirmed that a light-emitting device which is prepared by using a composition containing a compound (C-4) and a phosphorescent compound is excellent in luminous efficiency.

### Example 7

The value of lowest triplet excitation energy T₁ of a compound (C-5) represented by the following formula:

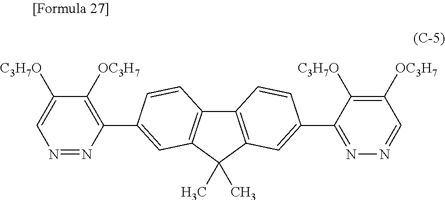

was 2.8 eV and an absolute value E_{LUMO} of an LUMO energy level was 1.9 eV. Note that the value of the lowest triplet excitation energy T₁ and an absolute value of an LUMO energy level were calculated by the computational scientific approach in the same manner as in Example 3.

It can be confirmed that a light-emitting device which is prepared by using a composition containing the compound (C-5) and a phosphorescent compound, is excellent in luminous efficiency.

### Example 8

The value of lowest triplet excitation energy T₁ of a compound (C-6) represented by the following formula:

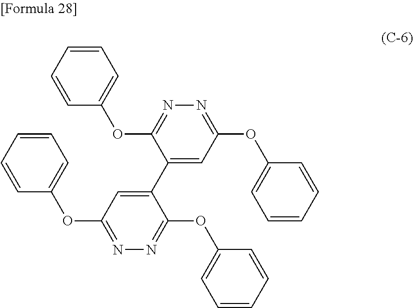

was 2.9 eV and an absolute value E_{LUMO} of an LUMO energy level was 2.5 eV. Note that the value of the lowest triplet excitation energy T₁ and an absolute value of an LUMO energy level were calculated by the computational scientific approach in the same manner as in Example 3.

It can be confirmed that a light-emitting device which is prepared by using a composition containing the compound (C-6) and a phosphorescent compound is excellent in luminous efficiency.

### Example 9

The value of lowest triplet excitation energy T₁ of a compound (C-7) represented by the following formula:

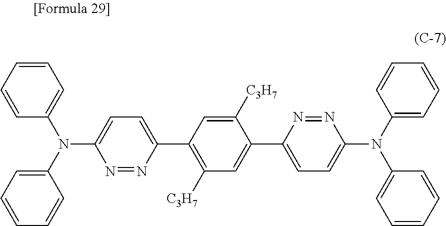

was 2.7 eV and an absolute value E_{LUMO} of an LUMO energy level was 1.7 eV. Note that the value of the lowest triplet excitation energy T₁ and an absolute value of an LUMO energy level were calculated by the computational scientific approach in the same manner as in Example 3.

It can be confirmed that a light-emitting device which is prepared by using a composition containing the compound (C-7) and a phosphorescent compound is excellent in luminous efficiency.

### Example 10

With a THF solution (0.05 wt %) of a phosphorescent compound (MC-1) synthesized by the method described in WO02/066552 and represented by the following formula:

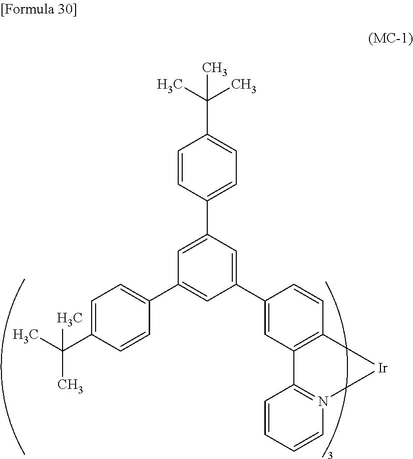

about a 5-fold weight of a THF solution (about 1 wt %) of a compound (C-8) represented by the following formula:

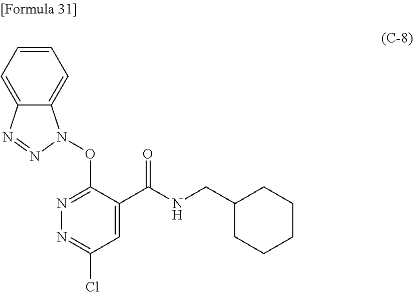

was mixed to prepare a mixture. This mixture (solution, 10 μl) was added dropwise to a slide glass and air-dried to obtain a solid film. When the solid film was irradiated with UV rays of 365 nm, strong green light was emitted from the phosphorescent compound (MC-1). From this, it was confirmed that the luminous efficiency of the mixture is high.

The T₁ energy value of the compound (C-8) was 2.9 eV and the absolute value E_{LUMO} of an LUMO energy level was 3.0 eV. Note that parameters were calculated by the computational scientific approach in the same manner as in Example 3.

Furthermore, the T₁ energy value (ETT) of the phosphorescent compound (MC-1) calculated by the computational scientific approach was 2.7 eV.

### Example 11

With a THF solution (0.05 wt %) of a phosphorescent compound (MC-1), about a 5-fold weight of a THF solution (about 1 wt %) of a compound (C-9) represented by the following formula:

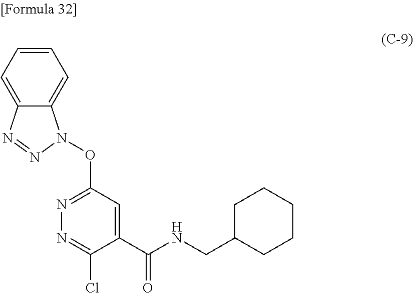

was mixed to prepare a mixture. This mixture (solution, 10 μl) was added dropwise to a slide glass and air-dried to obtain a solid film. When the solid film was irradiated with UV rays of 365 nm, strong green light was emitted from the phosphorescenct compound (MC-1). From this, it was confirmed that the luminous efficiency of the mixture is high.

The T₁ energy value of the compound (C-9) was 2.9 eV and the absolute value E_{LUMO} of an LUMO energy level was 2.9 eV. Parameters were calculated by the computational scientific approach in the same manner as in Example 3.

### Comparative Example 1

A macromolecular compound (P-3) represented by the following formula:

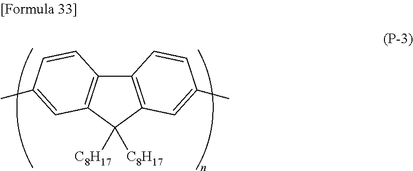

wherein, n is the number of polymerization units,
had a value of the lowest triplet excitation energy T₁ (1/n=0) of 2.6 eV, an absolute value E_{LUMO} (1/n=0) of a lowest unoccupied molecular orbital energy level of 2.1 eV, both of which were extrapolation values thereof at n=∞, and the smallest dihedral angle of 45°.

Parameters were calculated in the same manner as in Example 1 by using a simplified repeating unit (M-3) given below:

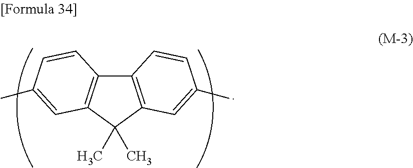

Subsequently, a mixture (10 μl) containing the macromolecular compound (P-3) and the phosphorescent compound (MC-1) was prepared, added dropwise to a slide glass and air-dried to obtain a solid film. When the solid film was irradiated with UV rays of 365 nm, weak light was emitted from the phosphorescent compound (MC-1). From this, it was confirmed that the luminous efficiency of the mixture is low.

1. A composition comprising a compound having a pyridazine ring structure and a phosphorescent compound.

2. The composition according to claim 1, wherein the compound having a pyridazine ring structure is a compound having at least one pyridazine ring structure selected from the group consisting of pyridazine ring structures represented by general formulas (1-1), (1-2), (2-1), (2-2), (2-3) and (2-4) given below: 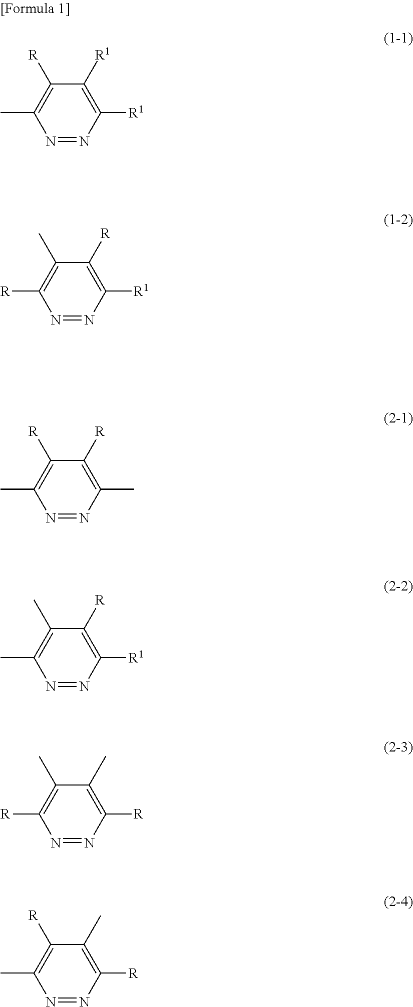
wherein R and R¹ each independently represent a hydrogen atom or a monovalent substituent; and when there are more than one R or R¹, they may be the same or different.

3. The composition according to claim 1, wherein the values of the lowest triplet excitation energy of the compound having a pyridazine ring structure as calculated by a computational scientific approach is 2.7 eV or more.

4. The composition according to claim 1, wherein the absolute value of the lowest unoccupied molecular orbital energy level of the compound having a pyridazine ring structure as calculated by a computational scientific approach is 1.5 eV or more.

5. The composition according to claim 1, wherein the compound having a pyridazine ring structure is a compound represented by general formula (3-1) or (3-2) given below:
[Formula 2]
pdz-(Y¹)ₙ—Ar¹ (3-1)
pdz-(Y¹)ₙ-pdz (3-2)
wherein pdz represents a pyridazine ring structure represented by general formula (1-1) or (1-2) given above; when there is more than one, they may be the same or different; Y¹ represents —C(R^{a})(R^{b})—, —C(═O)—, N(R^{c})—, —O—, —Si(R^{d})(R^{e})—, R(R^{f})—, —S—, or —S(═O)₂—; n is an integer of 0 to 5; Ar¹ represents an aryl group that may have a substituent or a monovalent heterocyclic group that may have a substituent; when there is more than one Y¹, they may be the same or different; and R^{a}, R^{b}, R^{c}, R^{d}, R^{e} and R^{f} each independently represent a hydrogen atom or a monovalent substituent, or a compound having a residue of the foregoing compound.

6. The composition according to claim 1, wherein the compound having a pyridazine ring structure has a pyridazine ring structure represented by the general formula (1-1), (1-2), (2-1), (2-2), (2-3) or (2-4) given above and a partial structure having at least two π-conjugated electrons and being adjacent to the pyridazine ring structure, and the dihedral angle between the pyridazine ring structure and the partial structure is 20° or more.

7. The composition according to claim 2, wherein at least one of the R and R¹ is an alkyl group, an alkoxy group, an aryl group that may have a substituent or a hetero aryl group that may have a substituent.

8. The composition according to claim 2, wherein at least one of more than one R or R¹ is an alkyl group having 3 to 10 carbon atoms or an alkoxy group having 3 to 10 carbon atoms.

9. The composition according to claim 2, wherein at least one of the R is a monovalent substituent having 3 or more atoms in total except hydrogen atom.

10. The composition according to claim 1, wherein the value of the lowest triplet excitation energy (ETP) of the compound having a pyridazine ring structure and the value of the lowest triplet excitation energy (ETT) of the phosphorescent compound satisfy an expression given below:

11. The composition according to claim 1, wherein the compound having a pyridazine ring structure is a macromolecular compound.

12. A macromolecular compound having a residue of a phosphorescent compound and a pyridazine ring structure.

13. A light-emitting film prepared by using the composition according to claim 1.

14. An organic semiconductor film prepared by using the composition according to claim 1.

15. A light-emitting device prepared by using the composition according to claim 1.

16. A planar light source comprising the light-emitting device according to claim 15.

17. A segment display comprising the light-emitting device according to claim 15.

18. A dot matrix display comprising the light-emitting device according to claim 15.

19. A liquid crystal display comprising a light-emitting device according to claim 15 as a backlight.

20. A light comprising the light-emitting device according to claim 15.

21. A light-emitting film prepared by using the macromolecular compound according to claim 12.

22. An organic semiconductor film prepared by using the macromolecular compound according to claim 12.

23. A light-emitting device prepared by using the macromolecular compound according to claim 12.
